(19) United States
(12) Patent Application Publication
(10) Pub. No.: US 2025/0281705 A1
(43) Pub. Date: Sep. 11, 2025

(54) AN ENHANCER FOR DELIVERY OF AEROSOL FROM A PRESSURIZED METERED DOSE INHALER (PMDI)

(71) Applicant: AERODEL TECHNOLOGIES LLP, Navi Mumbai (IN)

(72) Inventors: Shradha SANGWAI, Navi Mumbai (IN); Zahir JAFFER, Mumbai (IN); Suraj Rajan K K, Pune (IN)

(73) Assignee: AERODEL TECHNOLOGIES LLP, Navi Mumbai (IN)

(21) Appl. No.: 18/707,643

(22) PCT Filed: Dec. 27, 2022
(86) PCT No.: PCT/IN2022/051130

§ 371 Date: May 6, 2024

(30) Foreign Application Priority Data
Dec. 30, 2021 (IN) 202121061896

(51) Int. Cl. A61M 15/00

(52) U.S. Cl. CPC A61M 15/009; A61M 15/0001; A61M 15/002; A61M 15/0086; A61M 2205/581

(57) ABSTRACT

The present subject matter discloses an enhancer (100) for delivery of aerosol from a pressurized Metered Dose Inhaler (pMDI, 10) to a user. The enhancer comprises a holding chamber (110), a connector (120) and a one way valve (130). The holding chamber (110) is having a first end (112) configured to receive aerosol from the pMDI (10) and a second end (114) configured to form a mouthpiece (114) for engagement with the user's mouth. The connector (120) is having an adapter end (122) configured to be connected with the pMDI (10) and a pivot end (124) configured to pivotally mount the first end (112) of the holding chamber (110). Further, the connector (120) is configured to swivel the holding chamber (110) around the pivot end (124).

### TECHNICAL FIELD

The present subject matter described herein, in general, relates to an enhancer for delivery of aerosol from a pressurized metered dose inhaler (pMDI) and more particularly to an enhancer configured to allow foldable configuration of the pMDI.

### BACKGROUND

Metered dose inhalers (MDI) are used for delivery of aerosol drug to a patient for treatment of various respiratory problems such as Asthma. Pressurized drug is released from the outlet of the MDI which is to be inhaled by the patient. However the amount of drug released doesn't match with the inhaling ability of the patient. Further the patient also has to match the timing of inhaling with the release of the drug which may not be possible for every patient. Therefore the pMDI is not efficient at delivering the drug and only delivers a part of the full dose released from the MDI container. Even if a patient times inhalation with the release of the drug from the pMDI, the amount of drug reaching the lungs is inconsistent, depending on factors such as how much of the drug is dispersed and entrained in the stream of air entering the patient's lungs and how much of the drug is deposited on surfaces of the inhalation apparatus and on the mouth of the patient. Further this kind of scenarios may lead to under-dosage or over-dosage and higher cost of the drugs. Therefore, synchronization of the breath and actuation of the pMDI is a challenge.

A solution to above problem is known by usage of a spacer connected to the pMDI which gives some time and space to the drug to spread for the patient to inhale as per their inhalation ability. Spacers help in case there is no synchronization. However, the spacers are available in big sizes and needs to be assembled before use which makes the overall device bulky and difficult to carry by the patient.

Therefore there is a requirement of an improved system or apparatus to deliver the drug or medicament from a pMDI to a patient with required efficiency while also being compact in size and easy to use without any assembly

### Object of the Invention

It is an object of the present invention to provide an enhancer for efficient delivery of aerosol from a pressurized metered dose inhaler (pMDI).

It is an object of the present invention to provide a compact yet more efficient device for delivery of required dose of aerosol to a patient.

It is an object of the present invention to provide a foldable device for better ergonomic and carrying experience.

It is an object of the present invention to provide an enhancer with locking mechanism at fully folded and fully unfolded configurations.

It is an object of the present invention to provide an adaptable connector between a holding chamber and the pMDI.

### SUMMARY

Before the present system and method are described, it is to be understood that this application is not limited to the particular machine or an apparatus, and methodologies described, as there can be multiple possible embodiments that are not expressly illustrated in the present disclosures. It is also to be understood that the terminology used in the description is for the purpose of describing the particular versions or embodiments only, and is not intended to limit the scope of the present application. This summary is provided to introduce aspects related to an enhancer for delivery of aerosol from a pressurized metered dose inhaler (pMDI) and the aspects are further elaborated below in the detailed description. This summary is not intended to identify essential features of the proposed subject matter nor is it intended for use in determining or limiting the scope of the proposed subject matter.

The present subject matter discloses an enhancer for delivery of aerosol from a pressurized Metered Dose Inhaler (pMDI) to a user. The enhancer comprises a holding chamber and a connector. The holding chamber is configured to have a first end configured to receive aerosol from the pMDI and a second end configured to form a mouthpiece for engagement with the user's mouth. The connector is configured to have an adapter end configured to be mounted with the pMDI and a pivot end configured to pivotally mount the first end of the holding chamber. Further, the connector is configured to swivel the holding chamber around the pivot end.

### STATEMENT OF THE INVENTION

The present subject matter discloses an enhancer for delivery of aerosol from a pressurized Metered Dose Inhaler (pMDI) to a user. The enhancer comprises a holding chamber and a connector. The holding chamber is having a first end configured to receive aerosol from the pMDI and a second end configured to form a mouthpiece for engagement with the user's mouth. The connector is having an adapter end configured to be mounted with the pMDI and a pivot end configured to pivotally mount the first end of the holding chamber. Further, the connector is configured to swivel the holding chamber around the pivot end. A one way valve is configured to be positioned inside the holding chamber to provide one way path for the aerosol from the first end to the second end of the holding chamber.

A screen having a plurality of apertures is configured between the first end and the second end of the holding chamber for disseminating the aerosol into fine particles uniformly from the first end to the mouthpiece. The one way valve and the screen may be configured to form an integrated part. In an embodiment, the screen is configured to be positioned before the one way valve in the path of aerosol. In an embodiment, the screen is configured to be positioned after the one way valve in the path of aerosol. The holding chamber is configured with a seating arrangement in proximity of the mouthpiece for positioning of the one way valve and the screen.

The holding chamber is configured to be formed from a first chamber and a second chamber. In an embodiment, the first chamber and the second chamber are configured to be joined by a snap fit mechanism. In an embodiment, the first chamber and the second chamber are configured to be joined by one of a telescopic arrangement or threads to vary the size and volume of the holding chamber.

The holding chamber is configured to swivel between a folded and unfolded configuration, wherein a swivel angle (a) between the holding chamber and the pMDI ranges between 0° and 120°.

In an aspect, the enhancer is configured with a locking mechanism configured to lock the enhancer in a folded configuration and a fully unfolded configuration. The locking mechanism comprises, at least one locking arm configured to be mounted on two opposing sides of the connector; at least one first groove on two opposing sides of the holding chamber configured to receive the locking arm in the folded configuration of the enhancer, and at least one second groove on two opposing sides of the holding chamber configured to receive the locking arm in the fully unfolded configuration of the enhancer. The locking arm is configured to move on a rounded edge to provide a controlled movement to the locking arm for movement between the first groove and the second groove.

Further, the locking mechanism is configured to generate a click sound on locking at the folded configuration and the fully unfolded configuration. Further, the holding chamber is configured with a volume in a range of 50 ml to 150 ml. Further, the connector is configured to be detachable from the pMDI. Further, the one way valve is configured to be a duckbill valve.

### BRIEF DESCRIPTION OF THE DRA WINGS

The foregoing summary, as well as the following detailed description of embodiments, is better understood when read in conjunction with the appended drawing. For the purpose of illustrating the disclosure, there is shown in the present document example constructions of the disclosure, however, the disclosure is not limited to the specific methods and apparatus disclosed in the document and the drawing:

The detailed description is described with reference to the accompanying figure. In figures, a reference number is used to illustrate an element or feature of the present subject matter. The same reference numbers are used throughout the drawing to refer like features and elements.

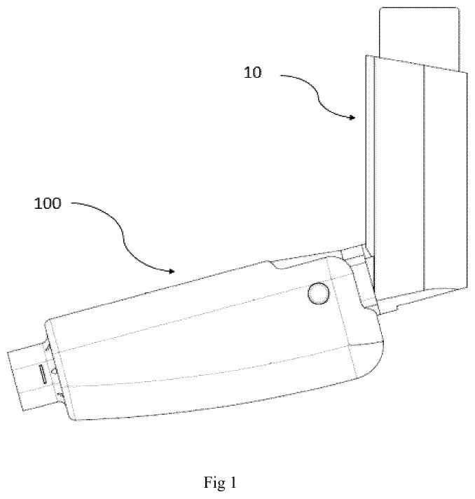

FIG. 1 illustrates a view of an enhancer for delivery of aerosol from a pressurized Metered Dose Inhaler (PMDI), in accordance with one embodiment of the present subject matter.

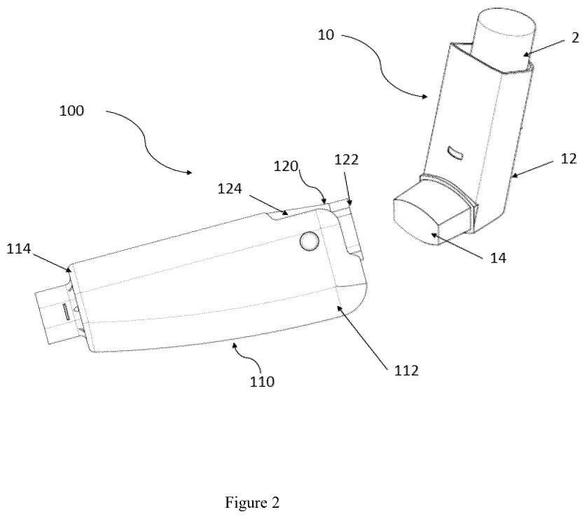

FIG. 2 illustrates another view of the enhancer with the pMDI, in accordance with one embodiment of the present subject matter.

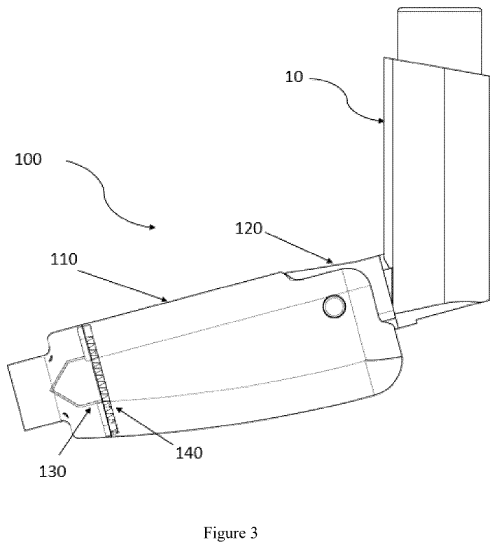

FIG. 3 illustrates another view of the enhancer with the pMDI, in accordance with one embodiment of the present subject matter.

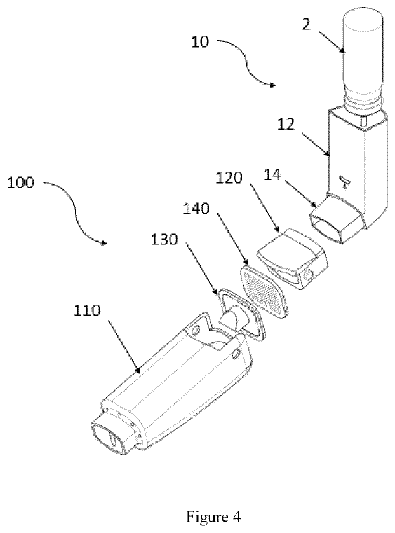

FIG. 4 illustrates an exploded view of the enhancer and the pMDI, in accordance with another embodiment of the present subject matter.

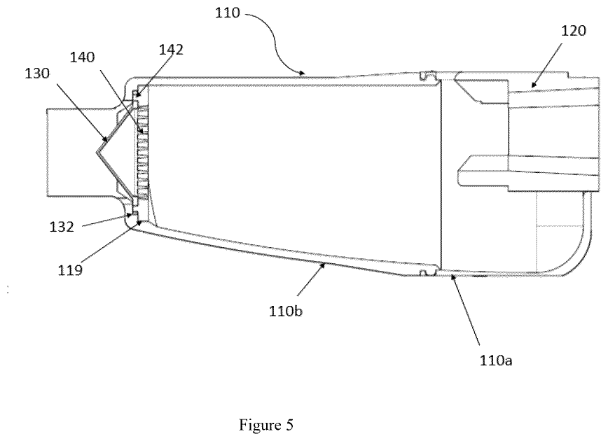

FIG. 5 illustrates a section view of the enhancer, in accordance with one embodiment of the present subject matter.

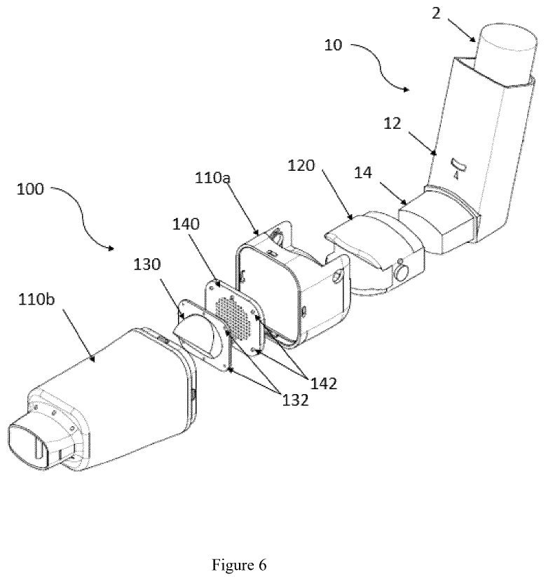

FIG. 6 illustrates another exploded view of the enhancer and the pMDI, in accordance with one embodiment of the present subject matter.

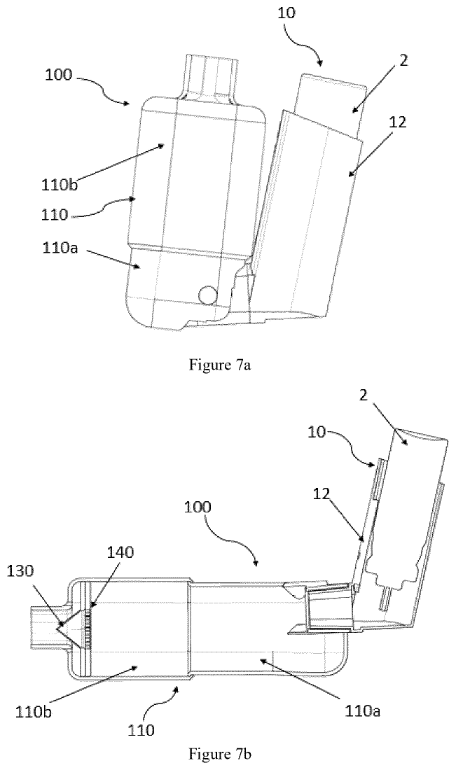

FIG. 7a illustrates a view of the enhancer with the holding chamber in collapsed configuration, in accordance with one embodiment of the present subject matter.

FIG. 7b illustrates a view of the enhancer with the holding chamber in expanded configuration, in accordance with one embodiment of the present subject matter.

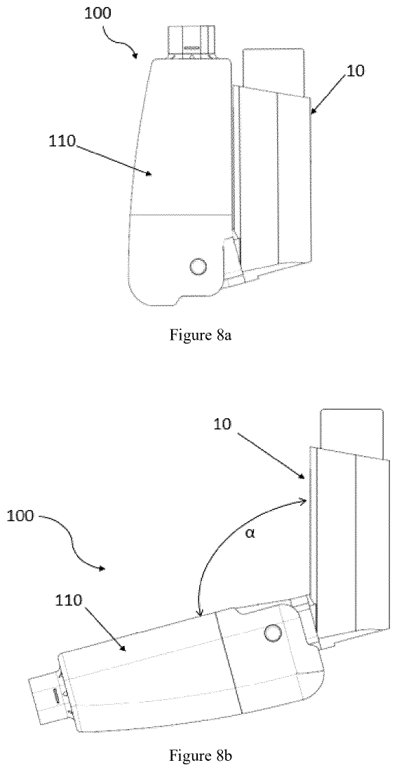

FIG. 8a illustrates a view of the enhancer in fully folded configuration, in accordance with one embodiment of the present subject matter.

FIG. 8b illustrates a view of the enhancer in fully unfolded configuration, in accordance with one embodiment of the present subject matter.

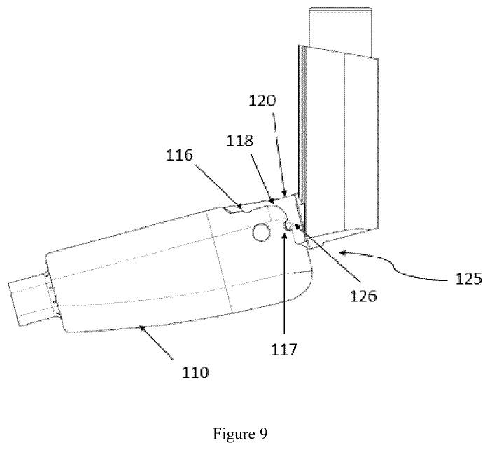

FIG. 9 illustrates another view of the enhancer with a locking mechanism, in accordance with one embodiment of the present subject matter.

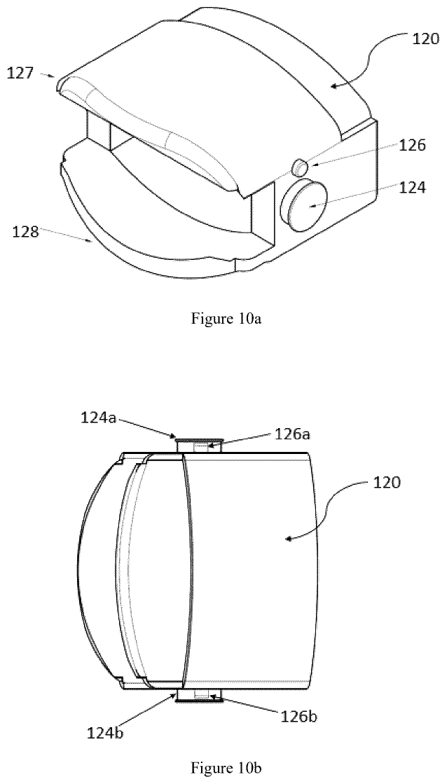

FIG. 10a illustrates an isometric view of a connector with the locking mechanism, in accordance with one embodiment of the present subject matter.

FIG. 10b illustrates a top view of the connector with the locking mechanism, in accordance with one embodiment of the present subject matter.

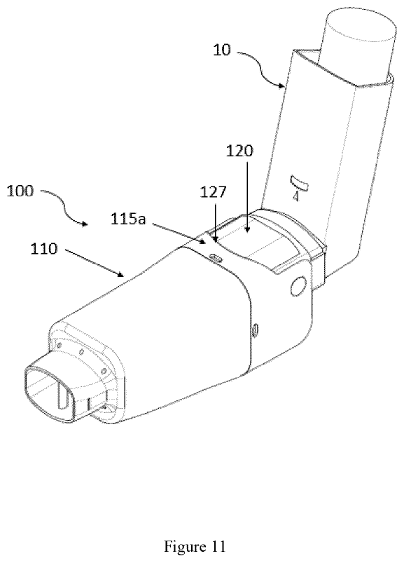

FIG. 11 illustrates an isometric view of the enhancer showing sealing at a first surface of the holding chamber, in accordance with one embodiment of the present subject matter.

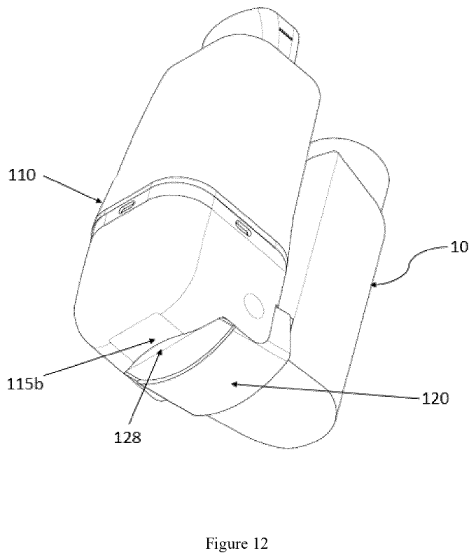

FIG. 12 illustrates an isometric view of the enhancer showing sealing at a second surface of the holding chamber, in accordance with one embodiment of the present subject matter.

The figure depicts various embodiments of the present disclosure for purposes of illustration only. One skilled in the art will readily recognize from the following discussion that alternative embodiments of the structures and methods illustrated herein may be employed without departing from the principles of the disclosure described herein.

### DETAILED DESCRIPTION

Some embodiments of this disclosure, illustrating all its features, will now be discussed in detail. The words “comprising”, “having”, and “including,” and other forms thereof, are intended to be equivalent in meaning and be open ended in that an item or items following any one of these words is not meant to be an exhaustive listing of such item or items, or meant to be limited to only the listed item or items. Although any systems and methods similar or equivalent to those described herein can be used in the practice or testing of embodiments of the present disclosure, the exemplary, systems and methods are now described. The disclosed embodiments are merely exemplary of the disclosure, which may be embodied in various forms.

Various modifications to the embodiment will be readily apparent to those skilled in the art and the generic principles herein may be applied to other embodiments. However, one of ordinary skill in the art will readily recognize that the present disclosure is not intended to be limited to the embodiments illustrated, but is to be accorded the widest scope consistent with the principles and features described herein.

Below is a list of elements with reference numerals which may be used for implementation of the present subject matter.

| Reference Numerals | Element name |
|---|---|
| 2 | Canister |
| 10 | pMDI |
| 12 | Holder |
| 14 | Outlet |
| 100 | The enhancer |
| 110 | holding chamber |
| 110a | Frist chamber |
| 110b | Second chamber |
| 112 | First end of the holding |
| | chamber |
| 114 | Second end of the holding |
| | chamber/mouthpiece |
| 115a | First surface of the holding |
| | chamber |
| 115b | Second surface of the holding |
| | chamber |
| 116a, 116b | First groove |
| 117a, 117b | Second groove |
| 118 | Rounded edge |
| 119 | Seating arrangement |
| 120 | Connector |
| 122 | Adapter end |
| 124a, 124b | Pivoting arms |
| 124 | Pivot end |
| 126a, 126b | Locking arms |
| 127 | First edge |
| 128 | Second edge |
| 130 | One way valve |
| 132 | Grooves on one way valve |
| 140 | Screen |
| 142 | Push locks |
| α | Swivel angle |
| 125 | Locking mechanism |

Referring now to FIG. 1, an enhancer 100 for delivery of aerosol from a pressurized metered dose inhaler (pMDI, 10) is illustrated. The enhancer 100 is configured to be fluidically connected with the pMDI to receive the aerosol.

Referring now to FIG. 2, the enhancer 100 comprises a holding chamber 110 and a connector 120. The holding chamber 110 is configured to have a first end 112 and a second end 114. The first end 112 is configured to receive aerosol from the pMDI10 and the second end 114 is configured to form a mouthpiece for engagement with the user's mouth. The connector 120 is configured to have an adapter end 122 and a pivot end 124. The adapter end 122 is configured to be connected with the pMDI 10 and the pivot end 124 is configured to pivotally mount the first end 112 of the holding chamber 110. In an embodiment, the adapter end 122 is configured to have tapered entry making it compactable with multiple types of pMDIs. Further in another embodiment, the connector may be configured with an elastomeric material for better fitment on multiple pMDIs. Further the connector 120 is configured to swivel the holding chamber 110 around the pivot end 124. A one way valve 130 is configured to be positioned inside the holding chamber 110 to provide one way path for the aerosol from the first end 112 to the second end/mouthpiece 114 of the holding chamber 110. The one way valve 130 is configured to hold the aerosol in the holding chamber 110 while the patient readies himself/herself to inhale the aerosol. However without the one way valve, the aerosol will reach the patient unprepared. Further the one way valve 130 is configured to stop/prevent the patient from exhaling/breathing into the enhancer 100 and without the one way valve the patient can breathe back into the holding chamber 110 and push the aerosol out through the pMDI.

The pMDI 10 is configured with a holder 12 to accommodate the canister 2. The canister 2 is configured to be filled with pressurized aerosol or any similar medicament. The canister 2 has to be pushed from the top to release the aerosol from an outlet 14 of the pMDI 10. The outlet 14 is configured to be fluidically connected with the adapter end 122 of the connector 120 for delivering the aerosol into the holding chamber 110.

As illustrated in FIG. 3, in one aspect of the present subject matter, the enhancer 100 is further configured with a screen 140 provided between the first end 112 and the second end 114 of the holding chamber 110. The screen 140 is configured with a plurality of apertures for disseminating the aerosol into fine particles uniformly from the first end 112 to the mouthpiece 114. Further, the FIG. 4 illustrates an exploded view of the enhancer 100 and the pMDI 10.

As illustrated in FIGS. 5 and 6, in one aspect of the present subject matter, the one way valve 130 and the screen 140 are configured to form an integrated part. The one way valve 130 is configured with one or more grooves 132 for receiving one or more push locks 142 of the screen 140. Further the holding chamber 110 is configured with a seating arrangement 119 for positioning of the integrated part containing the one way valve 130 and the screen 140. In an embodiment, the holding chamber 110 is configured with the seating arrangement 119 in proximity of the mouthpiece 114 for positioning of the one way valve 130 and the screen 140.

In one aspect of the present subject matter, the screen 140 is configured to be positioned before the one way valve 130 in the path of aerosol. In another aspect of the present subject matter, the screen 140 is configured to be positioned after the one way valve 130 in the path of aerosol.

In one aspect of the present subject matter, the holding chamber 110 is configured to be formed from a first chamber 110a and a second chamber 110b. The first chamber 110a and the second chamber 110b are configured to be joined by a snap fit mechanism.

In an embodiment as illustrated in FIGS. 7a and 7b, the first chamber 110a and the second chamber 110b are configured to be joined by one of a telescopic arrangement or threads to vary the size and volume of the holding chamber 110. The FIG. 7a illustrates a collapsed configuration of the holding chamber 110 resulting in a very compact version of the enhancer 100 for storage or carrying purposes. The FIG. 7a illustrates an expanded configuration of the holding chamber 110 for providing increased space for the aerosol during the usage of the enhancer 100. The size and volume of the holding chamber 110 can be varied based on the type of the medicament and based on the requirement of the patient.

FIGS. 8a and 8b illustrate a folded and unfolded configuration of the enhancer 100. In one aspect of the present subject matter, the holding chamber 110 is configured to swivel between a folded and unfolded configuration. In an embodiment, a swivel angle α between the holding chamber 110 and the pMDI 10 ranges between 0° and 120°. The enhancer 100 can be used in the unfolded configuration and can be stored in purse, pocket etc. in the folded configuration for convenient handling. The enhancer 100 is not required to be detached from the pMDI during storage or carrying in pockets whereas, the enhancer provides an always connected design configured to be stored in the folded position with the pMDI.

As illustrated in FIG. 9, in one aspect of the present subject matter, the enhancer 100 is configured with a locking mechanism 125 configured to lock the enhancer in a folded configuration and a fully unfolded configuration. The locking mechanism 125 comprises at least one locking arm 126 configured to be mounted on two opposing sides of the connector 120, at least one first groove 116 on two opposing sides of the holding chamber 110 configured to receive the locking arm 126 in the folded configuration of the enhancer 100 and at least one second groove 117 on two opposing sides of the holding chamber 110 configured to receive the locking arm 126 in the fully unfolded configuration of the enhancer 100. Further, the locking arm 126 is configured to move on a rounded edge 118 to provide a controlled movement to the locking arm 126 for movement between the first groove 116 and the second groove 117.

In an embodiment, the locking mechanism 125 is configured to generate a click sound on locking at the folded configuration and the fully unfolded configuration. The click sound at the folded configuration communicates the user that the enhancer 100 is ready to be stored and the locking mechanism keeps the enhancer 100 locked during the storage. Similarly the click sound at the unfolded configuration communicates the user that the enhancer 100 is ready to be used and the locking mechanism keeps the enhancer 100 locked at the fully unfolded configuration.

The FIGS. 9a, 9b, 10 and 11 illustrate the details of the connector 120 comprising the locking arms (126a, 126b) and the pivoting arms (124a, 124b) on two opposing sides. Further the pivoting end 122 of the connector 120 is configured with a first edge 127 and a second edge 128. The first edge 127 is configured to abut with a first surface 115a of the holding chamber 110 to seal the enhancer 100 in the fully unfolded configuration of the enhancer as illustrated in FIG. 10. Similarly the second edge 128 is configured to abut with a second surface 115b of the holding chamber 110 to seal the enhancer 100 in the fully folded configuration of the enhancer as illustrated in FIG. 11.

Further in an embodiment of the present subject matter, the holding chamber 110 is configured with a volume in a range of 50 ml to 150 ml. Further in another embodiment of the present subject matter, the connector 120 is configured to be detachable from the pMDI. Further in another embodiment of the present subject matter, the one way valve 130 is configured to be a duckbill valve.

Further in another embodiment of the present subject matter, the inner volume of the holding chamber is optimized from the flow dynamics perspective using an antistatic chamber to stop the drug/aerosol from adhering to the walls of the holding chamber and helps divert the flow towards the center of the mouthpiece. The shape of the holding chamber is configured to be tapered and converging towards the second end of the holding chamber. Therefore, the shape of the holding chamber is designed to ensure optimized flow and maximize the volume available and avoid dead space in the holding chamber.

The present subject matter discloses an enhancer for delivery of aerosol from a pressurized Metered Dose Inhaler (pMDI) to a user. The enhancer comprises a holding chamber and a connector. The holding chamber is having a first end configured to receive aerosol from the pMDI and a second end configured to form a mouthpiece for engagement with the user's mouth. The connector is having an adapter end configured to be mounted with the pMDI and a pivot end configured to pivotally mount the first end of the holding chamber. Further, the connector is configured to swivel the holding chamber around the pivot end. A one way valve is configured to be positioned inside the holding chamber to provide one way path for the aerosol from the first end to the second end of the holding chamber.

A screen having a plurality of apertures is configured between the first end and the second end of the holding chamber for disseminating the aerosol into fine particles uniformly from the first end to the mouthpiece. The one way valve and the screen may be configured to form an integrated part. In an embodiment, the screen is configured to be positioned before the one way valve in the path of aerosol. In an embodiment, the screen is configured to be positioned after the one way valve in the path of aerosol. The holding chamber is configured with a seating arrangement in proximity of the mouthpiece for positioning of the one way valve and the screen.

The holding chamber is configured to be formed from a first chamber and a second chamber. In an embodiment, the first chamber and the second chamber are configured to be joined by a snap fit mechanism. In an embodiment, the first chamber and the second chamber are configured to be joined by one of a telescopic arrangement or threads to vary the size and volume of the holding chamber.

The holding chamber is configured to swivel between a folded and unfolded configuration, wherein a swivel angle (a) between the holding chamber and the pMDI ranges between 0° and 120°.

In an aspect, the enhancer is configured with a locking mechanism configured to lock the enhancer in a folded configuration and a fully unfolded configuration. The locking mechanism comprises, at least one locking arm configured to be mounted on two opposing sides of the connector; at least one first groove on two opposing sides of the holding chamber configured to receive the locking arm in the folded configuration of the enhancer, and at least one second groove on two opposing sides of the holding chamber configured to receive the locking arm in the fully unfolded configuration of the enhancer. The locking arm is configured to move on a rounded edge to provide a controlled movement to the locking arm for movement between the first groove and the second groove.

Further, the locking mechanism is configured to generate a click sound on locking at the folded configuration and the fully unfolded configuration. Further, the holding chamber is configured with a volume in a range of 50 ml to 150 ml. Further, the connector is configured to be detachable from the pMDI. Further, the one way valve is configured to be a duckbill valve.

Exemplary embodiments discussed above may provide certain advantages. Though not required to practice aspects of the disclosure, these advantages may include those provided by the following features.

Some object of the present invention enables to provide an enhancer for efficient delivery of aerosol from a pressurized metered dose inhaler (pMDI).

Some object of the present invention enables to provide a compact yet more efficient device for delivery of required dose of aerosol to a patient.

Some object of the present invention enables to provide a foldable device for better ergonomic and carrying experience

Some object of the present invention enables to provide an enhancer with locking mechanism at fully folded and fully unfolded configurations.

Some object of the present invention enables to provide an adaptable connector between a holding chamber and the pMDI.

1. An enhancer (100) for delivery of aerosol from a pressurized Metered Dose Inhaler (pMDI, 10) to a user, comprising;
a holding chamber (110) having; a first end (112) configured to receive aerosol from the pMDI (10) and a second end (114) configured to form a mouthpiece (114) for engagement with the user's mouth and
a connector (120) having; an adapter end (122) configured to be connected with the pMDI (10) and a pivot end (124) configured to pivotally mount the first end (112) of the holding chamber (110), wherein the connector (120) is configured to swivel the holding chamber (110) around the pivot end (124).

2. The enhancer for delivery of aerosol as claimed in claim 1, wherein a one way valve (130) is configured to be positioned inside the holding chamber (110) to hold the aerosol and to provide one way path for the aerosol from the first end (112) to the second end (114) of the holding chamber (110).

3. The enhancer for delivery of aerosol as claimed in claim 1, wherein a screen (140) having a plurality of apertures is configured between the first end (112) and the second end (114) of the holding chamber (110) for disseminating the aerosol into fine particles uniformly from the first end (112) to the mouthpiece (114).

4. The enhancer for delivery of aerosol as claimed in claim 1, wherein the one way valve (130) and the screen (140) are configured to form an integrated part.

5. The enhancer for delivery of aerosol as claimed in claim 1, wherein the screen (140) is configured to be positioned before the one way valve (130) in the path of aerosol.

6. The enhancer for delivery of aerosol as claimed in claim 1, wherein the screen (140) is configured to be positioned after the one way valve (130) in the path of aerosol.

7. The enhancer for delivery of aerosol as claimed in claim 1, wherein the holding chamber (110) is configured with a seating arrangement (119) in proximity of the mouthpiece (114) for positioning of the one way valve (130) and the screen (140).

8. The enhancer for delivery of aerosol as claimed in claim 1, wherein the holding chamber (110) is configured to be formed from a first chamber (110a) and a second chamber (110b).

9. The enhancer for delivery of aerosol as claimed in claim 1, wherein the first chamber (110a) and the second chamber (110b) are configured to be joined by a snap fit mechanism.

10. The enhancer for delivery of aerosol as claimed in claim 1, wherein the first chamber (110a) and the second chamber (110b) are configured to be joined by one of a telescopic arrangement or threads to vary the size and volume of the holding chamber (110).

11. The enhancer for delivery of aerosol as claimed in claim 1, wherein the holding chamber (110) is configured to swivel between a folded and unfolded configuration, wherein a swivel angle (α) between the holding chamber (110) and the pMDI (10) ranges between 0° and 120°.

12. The enhancer for delivery of aerosol as claimed in claim 1, wherein the enhancer (100) is configured with a locking mechanism (125) configured to lock the enhancer in a folded configuration and a fully unfolded configuration.

13. The enhancer for delivery of aerosol as claimed in claim 11, wherein the locking mechanism (125) comprises;
at least one locking arm (126) configured to be mounted on two opposing sides of the connector (120);
at least one first groove (116) on two opposing sides of the holding chamber (110) configured to receive the locking arm (126) in the folded configuration of the enhancer, and at least one second groove (117) on two opposing sides of the holding chamber (110) configured to receive the locking arm (126) in the fully unfolded configuration of the enhancer; wherein the locking arm (126) is configured to move on a rounded edge (118) to provide a controlled movement to the locking arm (126) for movement between the first groove (116) and the second groove (117).

14. The enhancer for delivery of aerosol as claimed in claim 11, wherein the locking mechanism (125) is configured to generate a click sound on locking at the folded configuration and the fully unfolded configuration.

15. The enhancer for delivery of aerosol as claimed in claim 1, wherein the holding chamber (110) is configured with a volume in a range of 50 ml to 150 ml.

16. The enhancer for delivery of aerosol as claimed in claim 1, wherein the connector (120) is configured to be detachable from the pMDI.

17. The enhancer for delivery of aerosol as claimed in claim 1, wherein the one way valve (130) is configured to be a duckbill valve.
